(19) United States
(12) Patent Application Publication
(10) Pub. No.: US 2025/0381400 A1
(43) Pub. Date: Dec. 18, 2025

(54) IMPLANTABLE SENSOR TRAINING

(71) Applicant: Cochlear Limited, Macquarie University, NSW (AU)

(72) Inventors: Zachary Mark SMITH, Pymble, NSW (AU); Adam HERSBACH, Richmond, VIC (AU); Christian KOLIND, East Killara, NSW (AU); Federico BOLNER, Antwerp (BE)

(21) Appl. No.: 18/878,388

(22) PCT Filed: Jun. 23, 2023
(86) PCT No.: PCT/IB2023/056511

§ 371 Date: Dec. 23, 2024

Related U.S. Application Data
(60) Provisional application No. 63/357,751, filed on Jul. 1, 2022.

(51) Int. Cl. A61N 1/36

(52) U.S. Cl. CPC A61N 1/36039

(57) ABSTRACT

Presented herein are methods for training a signal analysis module. The methods include generating an external audio input based on sound signals captured by one or more external microphones: generating, with the signal analysis module, an implantable audio input based on sound signals captured by one or more implantable microphones; analyzing the implantable audio input relative to the external audio input; and adjusting operation of the signal analysis module based on the analyzing of the implantable audio input relative to the external audio input

### BACKGROUND

### Field of the Invention

The present invention relates generally to implantable sensors, such as implantable microphones.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The first method comprises: generating an external audio input based on sound signals captured by one or more external microphones; generating, with a signal analysis module, an implantable audio input based on sound signals captured by one or more implantable microphones; analyzing the implantable audio input relative to the external audio input; and adjusting operation of the signal analysis module based on the analyzing of the implantable audio input relative to the external audio input.

In another aspect, a medica device is provided. The medical device comprises: one or more external sensors; one or more implantable sensors; a signal analysis module; and one or more processors, wherein the one or more processors are configured to: generate an external signal input based on signals captured by the one or more external sensors; generate, by the signal analysis module, an implantable signal input based on signals captured by the one or more implantable sensors; analyze the implantable signal input relative to the external signal input; and adjust operation of the signal analysis module based on the analyzing of the implantable signal input relative to the external signal input.

In another aspect, one or more non-transitory computer readable storage media are provided. The one or more non-transitory computer readable storage media comprise instructions that, when executed by a processor, cause the processor to: receive, at a machine-learning device, implantable sound signals captured by one or more implantable microphones of a hearing device, the machine-learning device being trained to transform the implantable sound signals to substantially match an external audio input generated based on external sound signals captured by one or more external microphones; process, by the machine-learning device, the implantable sound signals to generate implantable audio input; and output the implantable audio input to a recipient of the hearing device.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 2 is a block diagram of the cochlear implant system of FIG. 1A;

FIG. 3 is a flowchart illustrating a method for determining whether a hearing device is to operate in a training mode or the hearing device is to use a trained system to process audio input, in accordance with embodiments presented herein.

FIG. 4 is a block diagram illustrating a method of training a signal analysis module at an internal component of a hearing device, in accordance with embodiments presented herein.

FIG. 5 is a block diagram illustrating a method of training a signal analysis module at an external component of a hearing device, in accordance with embodiments presented herein.

FIG. 6 is a block diagram illustrating a method of training a signal analysis module at an external device in communication with a hearing device, in accordance with embodiments presented herein.

FIG. 7 is a flowchart illustrating a method of training a signal analysis module, in accordance with embodiments presented herein.

FIG. 8 is a flowchart illustrating a method of processing implantable sound signals using a signal analysis module, in accordance with embodiments presented herein; and

FIG. 9 is a schematic diagram illustrating a vestibular stimulator system with which aspects of the techniques presented herein can be implemented.

### DETAILED DESCRIPTION

Presented herein are techniques for improving the performance of implantable (subcutaneous) sensors, such as implantable microphones (e.g., subcutaneous microphones, middle ear microphones, oral cavity microphones, etc.). For example, in certain embodiments, machine learning or deep learning approaches, using neural networks with multiple hidden layers or Deep Neural Networks (DNNs), can be used to transform a signal from an implantable sensor to substantially match (e.g., more closely resemble) a signal received by an external sensor. For example, in the context of hearing devices, sounds and vibrations captured simultaneously by external microphones/sensors and implantable microphones/sensors can be used to train and/or update previously trained machine learning systems or neural networks that can be applied to (e.g., used to process) the signals captured by the implantable microphones/sensors.

When compared to traditional microphones used in hearing aids and cochlear implant sound processors, implantable (subcutaneous) microphones have unfavorable characteristics, such as high noise floor, non-flat frequency response, low sensitivity to external sounds, and high sensitivity to internal sounds/vibrations of the body (including heartbeat, breathing, and even vibrations induced from contralateral hearing aids). These unfavorable characteristics lead to relatively poorer speech understanding, sound quality, and satisfaction for recipients when using subcutaneous microphones. This problem is exacerbated in systems where the recipient can switch between (and thus compare) an external microphone (e.g., use of “external hearing”) and an implantable microphone (e.g., use of “invisible hearing”). These differences in sound/performance can increase over time due to each system changing in different ways (external microphone cover gets dirty, implantable microphone characteristics shift with biological changes such as growing, medical interventions, etc.), which could make the differences jarring each time the recipient switches between external and invisible hearing.

A machine learning system or neural network can be trained (e.g., using data from a large number of participants or using recipient-specific data) so that acoustic signals received at an implantable microphone can be transformed (processed) so as to closely resemble the acoustic signals received at an external microphone. More specifically, sounds and vibrations captured simultaneously by external sensors, such as an external microphone, and implantable sensors, such as an implantable microphone and/or implantable vibration sensor (e.g., accelerometer), can be used to train a “signal analysis module” that is used to generate an improved implantable microphone output signal (e.g., train a machine learning system and/or update a previously trained machine learning system to convert an internally captured sound signal into a signal that more closely resembles an externally captured sound signal). As described further below, in certain embodiments, the training can use population data from many comparisons to train a generic machine learning system for use in many recipients. In the same or other embodiments, the training can use recipient-specific data to train and/or update a system as it is being used in that recipient.

The signal analysis module (e.g., signal analysis machine learning system or neural network) can process acoustic signals (e.g., to remove unwanted signals from or transmitted by the body, to fill in missing information/frequency content (e.g., at low sound intensities and frequencies with a poor response), etc.) so that the sound quality of the invisible hearing mode more closely matches the sound quality of an external hearing mode before delivering it as sound in a hearing prosthesis. By continuously training and updating the signal analysis module, the external hearing and invisible hearing modes can produce similar sound quality despite changes in microphone characteristics that can occur.

In one embodiment, a single large machine learning system can be used to enhance the implantable microphone signal. In other embodiments, multiple machine learning systems can be deployed. In this scenario, a particular machine learning system can be selected by another machine learning algorithm (such as a DNN) that analyzes the current situation and selects the correct machine learning system to maximize performance for that situation (e.g., one machine learning system might be best for removing body noise, another might be best for soft external sounds, another might be best in noisy acoustic situations with multiple competing talkers).

At least some exemplary embodiments according to the teachings detailed herein utilize advanced learning signal processing techniques, which are trained to detect higher order, and/or non-linear statistical properties of signals. As discussed above, an exemplary signal processing technique is the DNN. At least some exemplary embodiments utilize a DNN (or any other advanced learning signal processing technique) to process a signal representative of captured sound, and the processed signal is utilized to evoke a hearing percept. At least some exemplary embodiments entail training signal processing algorithms to process signals indicative of captured sound. That is, some exemplary methods utilize learning algorithms or systems such as DNNs or any other system that would otherwise enable the teachings detailed herein to analyze captured sound.

A “neural network” is a specific type of machine learning system. Any disclosure herein of the species “neural network” constitutes a disclosure of the genus of a “machine learning system.” While embodiments herein focus on the species of a neural network, it is noted that other embodiments can utilize other species of machine learning systems accordingly, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

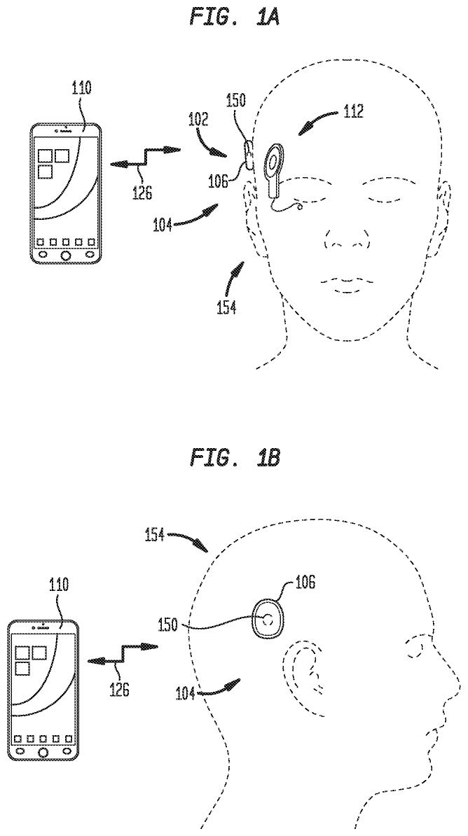

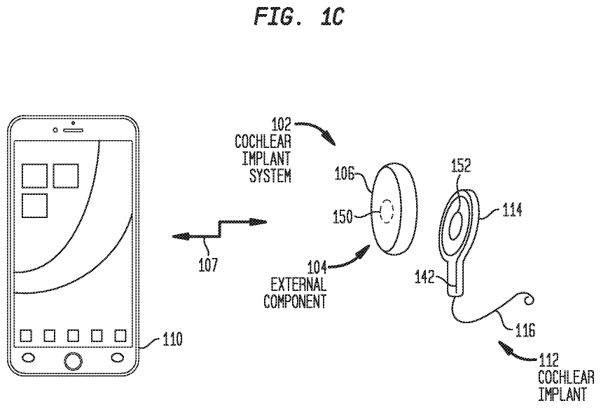

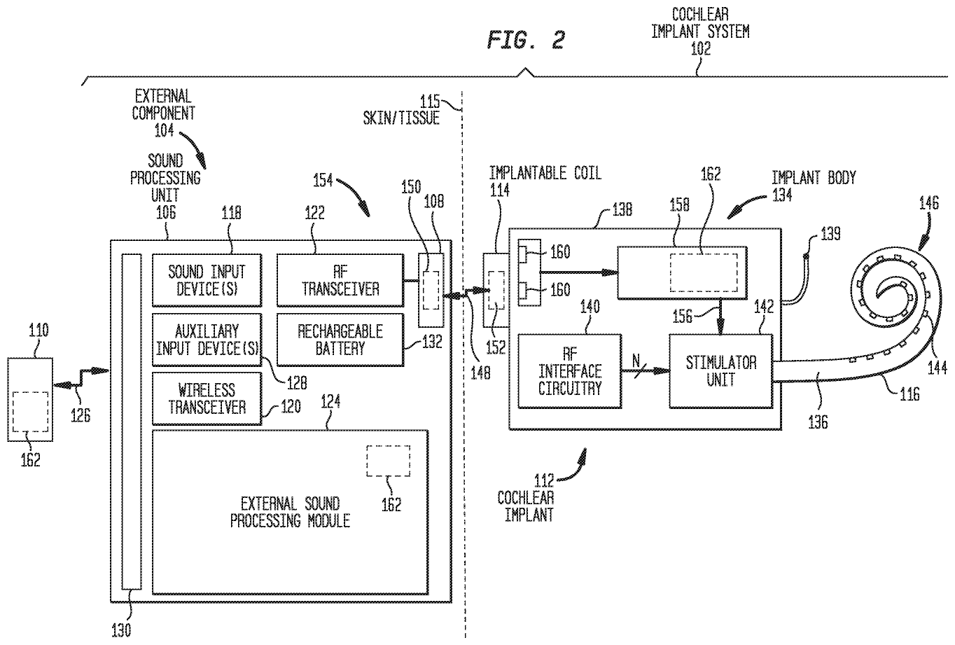

FIGS. 1A-1C and FIG. 2 illustrate an example cochlear implant system 102 with which aspects of the techniques presented herein can be implemented. The cochlear implant system 102 comprises an external component 104 and an implantable component 112. In these, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 2 illustrates further details of the cochlear implant system 102. For ease of description, FIGS. 1A, 1B, 1C, and 2 will generally be described together.

Cochlear implant system 102 includes an external component 104 that is configured to be directly or indirectly attached to the body of the recipient and an implantable component 112 configured to be implanted in the recipient. The external component 104 comprises a sound processing unit 106, while the cochlear implant 112 includes an implantable coil 114, an implant body 134, and an elongate stimulating assembly 116 configured to be implanted in the recipient's cochlea.

The sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external unit. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A, 1B, 1C, and FIG. 2, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or fewer input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 2).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 2). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 2 illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 2 illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 2, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 2, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

In the examples of FIGS. 1A, 1B, 1C, and FIG. 2, aspects of the techniques presented herein can be performed by one or more components of the cochlear implant system 102, such as the external sound processing module 124, the implantable sound processing module 158, and/or the external device 110, etc. This is generally shown by dashed boxes 162. That is, dashed boxes 162 generally represent potential locations for some or all of a “signal analysis module” 162, which is sometimes referred to herein as a “signal analysis machine-learning device” or as “signal analysis machine-learning logic” that, when executed, is configured to perform aspects of the techniques presented herein. As noted above, the external sound processing module 124, the implantable sound processing module 158, and/or the external device 110 can comprise, for example, one or more processors and a memory device (memory) that includes all or part of the signal analysis machine-learning device 162. The memory device can comprise any one or more of: NVM, RAM, FRAM, ROM, magnetic disk storage media devices, optical storage media devices, flash memory devices, electrical, optical, or other physical/tangible memory storage devices. The one or more processors are, for example, microprocessors or microcontrollers that execute instructions for the signal analysis machine-learning device 162 stored in a memory device. In some implementations, the signal analysis machine-learning device 162 can be a neural network or a DNN. As described further below, the signal analysis machine-learning device 162 can be implemented internally (e.g., implanted within the body of a recipient) and/or externally (e.g., outside the body of a recipient).

A signal analysis machine-learning device (signal analysis module) presented herein, such as signal analysis machine-learning device 162, is a functional block (e.g., one or more processors operating based on code, algorithm(s), etc.) that is trained, through a machine-learning process to transform sound signals captured by implantable sound sensors 160 into signals to be more like a sound signal captured and processed at external component 104. As described further below, the transformation of the sound signals captured by implantable sound sensors 160 into signals to be more like a sound signal captured and processed at external component 104 includes modelling and estimation of the external sound signal based on the implantable sound signal.

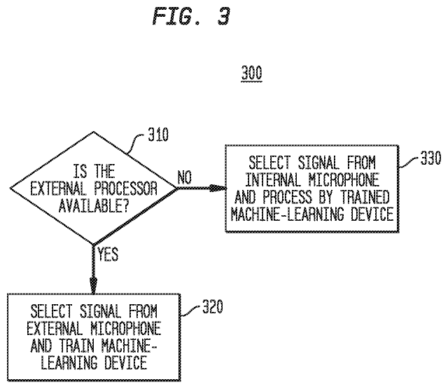

FIG. 3 is a flowchart of an example method 300 for determining whether a hearing device is to operate in a training mode or the hearing device is to use a trained system to process audio input, in accordance with embodiments presented herein. As described below, the signal analysis machine-learning device can be trained to process audio signals captured by an implantable microphone such that the audio signals output approximately match an output of the same audio signals captured by an external microphone of an external device. Training of the signal analysis machine-learning device can occur when both external and implantable microphone signals are simultaneously available.

Method 300 begins at 310 by determining whether an external processor is available. For example, cochlear implant system 102 can determine whether the external processor is on, such as when a recipient is using external hearing or is charging an implanted battery with sound processing unit 106. In some embodiments, cochlear implant system 102 can additionally determine whether the external processor is receiving the best signal. For example, cochlear implant system 102 can determine whether sound processing unit 106 is receiving clear audio signals.

If the external processor is available (and, optionally, the sound processor is receiving the best signal), at 320, a signal is selected from an external microphone and used to train a signal analysis machine-learning device. For example, if the sound processing unit 106 is on and receiving clear audio signals (e.g., at sound input devices 118), the cochlear implant system 102 can enter training mode and the audio signals captured by an external microphone can be used in conjunction with audio signals captured by one or more implantable microphones to train signal analysis machine-learning device 162. As described below, the training of the signal analysis machine-learning device can take place at external device 110, external component 104, or cochlear implant 112.

If the external processor is not available (and/or the external processor is not receiving the best signal), at 330, a signal is selected from an internal or implanted component of cochlear implant system 102 and the signal is processed by the trained signal analysis machine-learning device. For example, if the sound processing unit 106 is not on or if an audio signal received at sound processing unit 106 is not clear, training may not be performed and the cochlear implant system 102 can operate in invisible hearing mode. In invisible hearing mode, audio signals can be detected/captured by implantable sound sensors 160 and processed by trained signal analysis machine-learning device 162.

As noted above, certain aspects presented herein use a signal analysis machine-learning device, sometimes referred to herein as a signal analysis module, to determine how to transform implantable audio input to more closely resemble external audio input. The signal analysis module is a functional block (e.g., one or more processors operating based on code, algorithm(s), etc.) that is trained, through a machine-learning process, to modify sound signals captured at implantable microphones to more closely resemble external audio input generated based on sound signals captured at external microphones. In certain examples, the signal analysis machine-learning device, sometimes referred to as signal analysis module 362, is configured to estimate the external audio signal from the implantable audio signal using a trained model. In some embodiments, signal analysis module 362 can contain a neural network or a DNN and a facility for training the network.

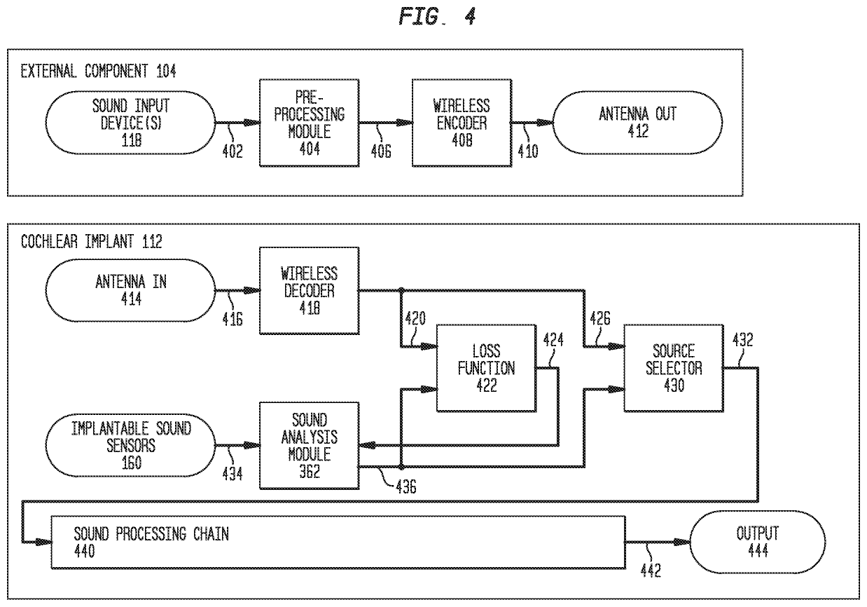

FIG. 4 is a functional block diagram illustrating the training of signal analysis module 362 at cochlear implant 112 in accordance with embodiments presented herein. As shown, sound input device(s) 118 of external component 104 capture a sound signal and, at 402, the sound signal is outputted to pre-processing module 404. Pre-processing module 404 can process the sound signal to generate an external audio signal by, for example, reducing noise, removing the noise floor, improving microphone directionality, removing or reducing unwanted sound (e.g., wind noise, very high signals in saturation, etc.), etc. At 406, the external audio signal is outputted to wireless encoder 408 for encoding and, at 410, wireless encoder 408 outputs the external audio signal to antenna out 412 for transmission to cochlear implant 112.

At cochlear implant 112, antenna in 414 receives the external audio signal from antenna out 412 of external component 104 and, at 416, transmits the external audio signal to wireless decoder 418 for decoding. At 420, wireless decoder 418 outputs the external audio signal.

Additionally, at cochlear implant 112, implantable sound sensors 160 capture the sound signal that was received at sound input device(s) 118 of external component 104 (e.g., at approximately the same time as sound input device(s) 118 captured the sound signal). At 434, implantable sound sensors 160 transmit the implantable sound signal to signal analysis module 362 for processing. Signal analysis module 362 has been trained to receive an implantable sound signal from implantable sound sensors 160 and transform the implantable sound signal to more closely match an external audio signal generated at external component 104 based on a sound signal captured by sound input device(s) 118. This transformation of the implantable sound signal can also be thought of a model and estimation of the external sound signal based on the implantable sound signal.

Signal analysis module 362 processes the implantable sound signal and, at 436, outputs an estimate of the external audio signal. Loss function 422 receives the estimate of the external audio signal from signal analysis module 362 and the actual external audio signal from wireless decoder 418. Loss function 422 analyzes the estimate of the external audio signal relative to the actual external audio signal. For example, loss function 422 compares the estimate of the external audio signal to the actual external audio signal and computes an error function to identify any differences between the two signals. At 424, loss function 422 outputs an indication of the difference between the two signals.

As discussed above, signal analysis module 362 has been trained to estimate as closely as possible the external audio signals from the implantable audio signals. Therefore, the differences between the implantable audio signal and the external audio signal that are identified by loss function 422 can be used to train and adjust parameters of the signal analysis module 362. As signal analysis module 362 continues to be trained, the estimates of the external audio signal based on the implantable audio signal can continue to more closely match the actual external audio signal.

Signal analysis module 362 receives the output of loss function 422 and adjusts operation based on the output of loss function 422. For example, signal analysis module 362 can set or update weights associated with signal analysis module 362 to improve processing of implantable audio signals to better model and estimate external audio signals. By adapting new or updated weights during training, signal analysis module 362 can continue to improve processing of the implantable sound signals so its estimates of external audio signals more closely match the actual external audio signals.

When the hearing device is in an external hearing mode, a recipient of the hearing device receives the external audio signals and when the hearing device is in invisible mode, the recipient receives the estimates of external audio signals from the implantable audio signals. Therefore, source selector 430 of cochlear implant 112 can receive the external audio signal from wireless decoder 418 and the implantable audio signal from signal analysis module 362. Source selector 430 can determine which signal to select to be outputted to the recipient of the cochlear implant system 102 based on the mode associated with cochlear implant system 102. When the cochlear implant system 102 is in external hearing mode, source selector 430 selects the external audio signal and when the cochlear implant system 102 is in invisible mode, source selector 430 selects the estimated external audio signal from the implantable audio signal. When a recipient switches a mode associated with the cochlear implant system 102, source selector 430 switches the selected audio signal. At 432, source selector 430 outputs the selected audio signal.

Sound processing chain 440 receives the selected audio signal. Sound processing chain 440 can include, for example, a filterbank, an envelope extraction module, a channel selection module, a loudness mapping module, and/or additional sound processing modules. Sound processing chain 440 generally operates to convert received sound signals into output signals, which can be used for delivering stimulation to a recipient in a manner that evokes perception of the sound signals. At 442, sound processing chain 440 transmits the processed sound signal to output 444 for delivering the stimulation to the recipient to evoke perception of the sound signal.

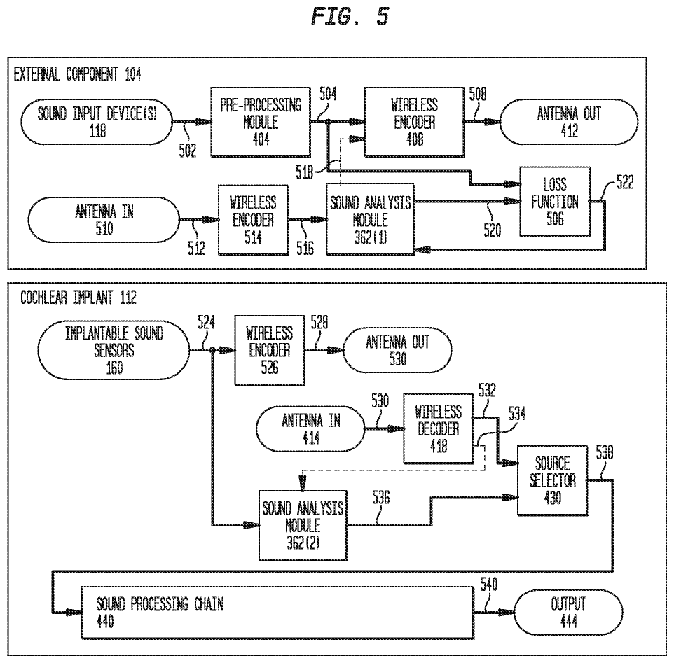

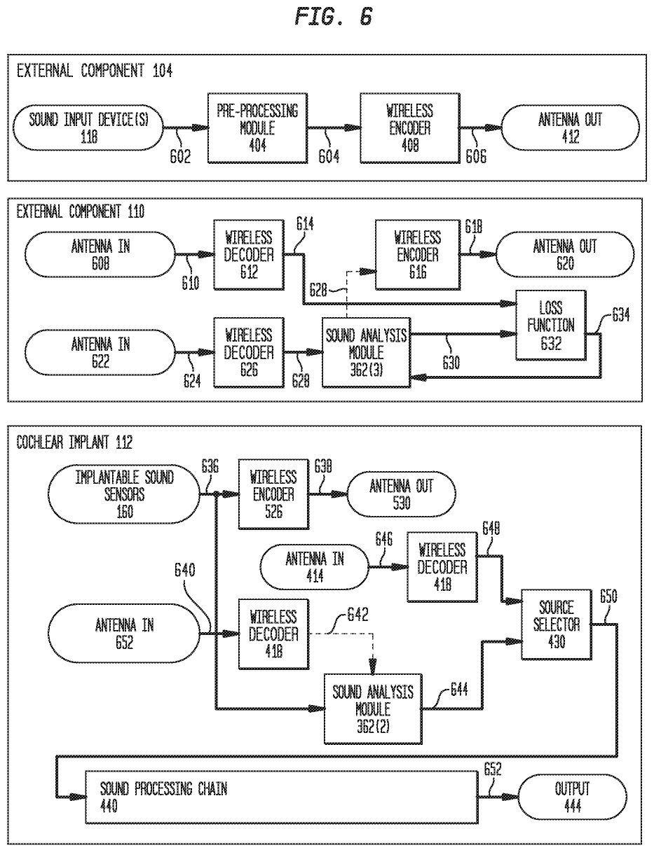

FIG. 5 is a functional block diagram illustrating the training of signal analysis module 362 at external component 104 in accordance with embodiments presented herein. In the example described with respect to FIG. 5, signal analysis module 362(1) is located at external component 104 and is trained when the hearing device is in training mode. Signal analysis module 362(2) at cochlear implant 112 receives information from the training of signal analysis module 362(1) and is used for processing sound signals captured by implantable sound sensors 160.

As shown, sound input device(s) 118 of external component 104 can capture a sound signal and, at 502, sound input device(s) 118 can output the sound signal to pre-processing module 404 for pre-processing in a manner similar to the manner described above with respect to FIG. 4. Pre-processing module 404 pre-processes the external sound signal and, at 504, pre-processing module 404 outputs an external audio signal.

At cochlear implant 112, implantable sound sensors 160 capture the sound signal (e.g., at approximately the same time as sound input device(s) 118 capture the sound signal) and, at 524, implantable sound sensors 160 output the implantable sound signal. Wireless encoder 525 encodes the implantable sound signal and, at 528, wireless encoder 525 transmits the implantable sound signal to antenna out 530 for transmission to external component 104.

At external component 104, antenna in 510 receives the implantable sound signal from antenna out 530 of cochlear implant 112, and, at 512, transmits the implantable sound signal to wireless decoder 514. Wireless decoder 514 decodes the implantable sound signal and, at 516, outputs the implantable sound signal. Signal analysis module 362(1) processes the implantable sound signal, as described above with respect to FIG. 4, to modify the implantable sound signal to more closely match an external audio signal and to generate an estimate of the external audio signal. At 520, signal analysis module 362(1) outputs the estimate of the external audio signal.

Loss function 506 receives the estimate of the external audio signal from signal analysis module 362(1) and the actual external audio signal from pre-processing module 404. As described above with respect to FIG. 4, loss function 506 analyzes the estimate of the external audio signal with respect to the actual external audio signal and computes an error function and/or identifies differences between the two audio signals. At 538, loss function 506 outputs an indication of the difference between the estimate of the external audio signal and the actual external audio signal. As described above with respect to FIG. 4, signal analysis module 362(1) can set or update weights based on the differences between the estimate of the external audio signal and the actual external audio signal.

Although the training of the signal analysis module 362(1) of FIG. 5 occurs at external component 104, signal analysis module 362(2) of cochlear implant 112 uses the new or updated weights for improved processing of sound signals captured at implantable sound sensors 160 (e.g., when the hearing device is in invisible mode). To transmit the new or updated weights to signal analysis module 362(2), wireless encoder 408 receives the new/updated weights from signal analysis module 362(1), encodes the weights and, at 508, transmit the encoded weights to antenna out 412 for transmission to cochlear implant 112.

At cochlear implant 112, antenna in 414 can receive the new/updated weights from antenna out 412. At 530, antenna in 414 outputs the new/updated weights to wireless decoder 418 for decoding and, at 534, wireless decoder 418 transmits the new/updated weights to signal analysis module 362(2) of cochlear implant 112. After receiving the new/update weights, signal analysis module 362(2) of cochlear implant 112 can provide improved processing of subsequent sound signals received at implantable sound sensors 160.

To provide audio signals to a recipient of the hearing device, external component 104 can additionally transmit the external audio signal to cochlear implant 112. To perform the transmission, wireless encoder 408 of external component 104 encodes the external audio signal and, at 508, outputs the external audio signal to antenna out 412 for transmission to cochlear implant 112. Antenna in 414 can receive the external audio signal from antenna out 412 and, at 530, antenna in 414 transmits the external audio signal to wireless decoder 418. Wireless decoder 418 can decode the external audio signal and, at 532, output the external audio signal.

Signal analysis module 362(2) receives the sound signal from implantable sound sensors 160 and processes the sound signals based on previous training. For example, signal analysis module 362(2) processes the sound signals output at implantable sound sensors 160 to resemble the sound signals that would have been output by pre-processing module 404 if the sound input device(s) 118 had received the same sound input. At 536, signal analysis module 362(2) outputs the processed sound signals.

Source selector 430 receives external audio signals from wireless decoder 418 and the estimates of external audio signals from the implantable audio signals from signal analysis module 362(2) and, at 538, outputs the external or the estimates of external audio signals from the implantable audio signals, as described above with respect to FIG. 4. For example, source selector 430 outputs the estimates of the external audio signals from the implantable audio signals when the hearing device is in invisible mode and outputs the external audio signals when the hearing device is in external hearing mode. Based on the source selection, a recipient of cochlear implant system 102 receives a processed sound signal either from sound input device(s) 118 of external component 104 or implantable sound sensor 160 of cochlear implant 112. At 538, source selector 430 transmits the selected audio signal to sound processing chain 440 and sound processing chain 440 processes the audio signal as described above with respect to FIG. 4. At 538, sound processing chain 440 transmits processed audio signal to output 444 for delivering the stimulation to the recipient to evoke perception of the sound signal.

FIG. 6 is a functional block diagram illustrating the training of signal analysis module 362 at external device 100 in accordance with embodiments presented herein. In the example described with respect to FIG. 6, a first communications link is provided between external component 104 and external device 110 and a second communications link is provided between external device 110 and cochlear implant 112. In addition, in the example described with respect to FIG. 6, signal analysis module 362(3) is located at external device 110 and is trained when the hearing device is in training mode. Signal analysis module 362(2) at cochlear implant 112 receives information related to the training of signal analysis module 362(3) that is used for processing sound signals captured by implantable sound sensors 160.

As shown, at external component 104, sound input device(s) 118 capture a sound signal and, at 602, output the external sound signal to pre-processing module 404 for pre-processing in a manner similar to the manner described above with respect to FIG. 4. At 604, pre-processing module 404 outputs the external audio signal to wireless encoder 408 for encoding, as described above with respect to FIG. 4. At 606, wireless encoder 408 outputs the external audio signal to antenna out 412 for transmission to cochlear implant 112 (e.g., when the hearing device is in external hearing mode) and to external device 110 for training signal analysis module 362(3).

At cochlear implant 112, implantable sound sensors 160 capture the sound signal (e.g., at approximately the same time as sound input device(s) 118 capture the sound signal) and, at 636, output the implantable sound signal. Wireless encoder 526 receives the implantable sound signal and encodes the implantable sound signal. At 638, wireless encoder 526 outputs the implantable sound signal to antenna out 530 for transmission to external device 110 for training signal analysis module 362(3).

At external device 110, antenna in 608 receives the external audio signal from antenna out 412 of external component 104 and, at 610, outputs the external audio signal to wireless decoder 612 for decoding. Wireless decoder 612 decodes the external audio signal and, at 614, outputs the external audio signal. Additionally, antenna in 622 receives the implantable sound signal from antenna out 530 of cochlear implant 112 and, at 624, antenna in 622 outputs the implantable sound signal to wireless decoder 626 for decoding. Wireless decoder 626 decodes the implantable sound signal and, at 628, outputs the implantable sound signal to signal analysis module 362(3) for processing. As described above, signal analysis module 362(3) has been trained to modify an implantable sound signal to generate an implantable audio signal that more closely resembles an external audio signal from external component 104. Signal analysis module 362(3) processes the implantable sound signal and, at 630, outputs an estimate of the external audio signal.

Loss function 632 receives the external audio signal from wireless decoder 612 and the estimate of the external audio signal from signal analysis module 362 and analyzes the actual external audio signal with respect to the estimate of the external audio signal. In particular, loss function 632 computes an error function and/or identifies differences between the two sound signals, as described above with respect to FIGS. 4 and 5. At 634, loss function 632 outputs an indication of the difference(s) between the two signals. Signal analysis module 362(3) receives an input identifying the difference(s) between the actual external audio signal and the estimate of the external audio signal and sets or updates weights/parameters for estimating as closely as possible the external audio signals from the implantable sound signals based on the difference(s). Signal analysis module 362(3) adjusts operations based on the new or updated weights/parameters. When the training of signal analysis module 362(3) is completed for the training mode session, at 628, signal analysis module 362(3) at external device 110 outputs the new or updated weights/parameters for transmission to the signal analysis module 362(2) at cochlear implant 112. Wireless encoder 616 encodes the new or updated weights/parameters and, at 614, outputs the encoded weights/parameters to antenna out 620 for transmission to cochlear implant 112.

At cochlear implant 112, antenna in 652 receives the new or updated weights/parameters from antenna out 412 and, at 640, outputs the new or updated weights/parameters to wireless decoder 418. Wireless decoder 418 decodes the new or updated weights/parameters and, at 642, transmits the new or updated weights/parameters to signal analysis module 362(2) at cochlear implant 112. By implementing the new or updated weights/parameters received from signal analysis module 362(3) at external device 110, signal analysis module 362(2) at cochlear implant 112 can improve processing of implantable sound signals to better model and estimate external audio signals based on the training of signal analysis module 362(3) at external device 110.

To provide the external audio signal to a recipient of a hearing device (e.g., when the hearing device is in external hearing mode), antenna in 414 at cochlear implant 112 receives the external audio signal from antenna out 412 and, at 646, outputs the signal to wireless decoder 418. Wireless decoder 418 decodes the external audio signal, and, at 648, outputs the decoded external component sound signal.

Source selector 430 receives the external audio signal from wireless decoder 418 and the estimate of the external audio signal from signal analysis module 362(2) and selects a single audio signal based on a mode of the hearing device. For example, if the hearing device is in external hearing mode, the source selector 430 selects the external audio signal and if the hearing device is in invisible mode, source selector 430 selects the estimate of the external audio signal from the implantable audio signal. Based on the source selection, a recipient of cochlear implant system 102 receives an audio signal either from sound input device(s) 118 of external component 104 or implantable sound sensor 160 of cochlear implant 112. At 650, source selector 430 transmits the selected audio signal to sound processing chain 440 and sound processing chain 440 processes the audio signal as described above with respect to FIG. 4. At 652, sound processing chain 440 transmits processed sound signal to output 444 for delivering the stimulation to the recipient to evoke perception of the sound signal.

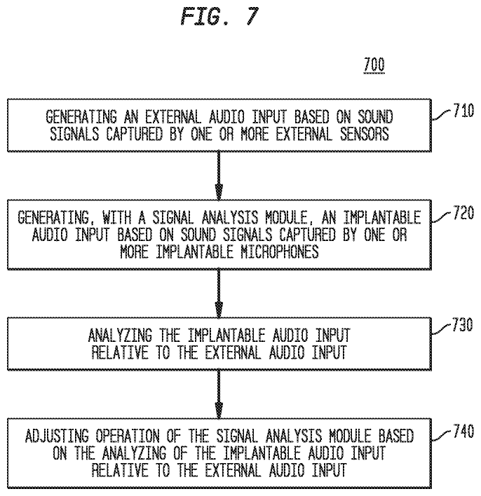

FIG. 7 is flowchart of an example method 700 of training a signal analysis module in accordance with certain embodiments presented herein. Method 700 can be performed at a hearing device system comprising a hearing device with an external component and an internal component. Method 700 can optionally be performed at one or more remote devices in communication with the hearing device, in accordance with certain embodiments presented herein.

Method 700 begins at 710 when an external audio input is generated based on sound signals captured by one or more external microphones. For example, sound input device(s) 118 can capture sound signals and external component 104 can generate an external audio input based on the sound signals. In some embodiments, generating the external audio input can include pre-processing the audio input (e.g., to remove noise, etc.).

At 720, a signal analysis module generates an implantable audio input based on sound signals captured by one or more implantable microphones. For example, implantable sound sensors 160 can capture sound signals and signal analysis module 362 can process the sound signals to generate an implantable audio input. In some embodiments, signal analysis module 362 can remove noise, fill in frequency content, or process the sound signals captured by the implantable sound sensors 160 in a different way.

At 730, the hearing system analyzes the implantable audio input relative to the external audio input. For example, the hearing system can determine differences between the implantable audio input and the external audio input. At 740, operations of the signal analysis module are adjusted based on the analyzing of the implantable audio input relative to the external audio input. For example, new or updated weights/parameters made be determined for the signal analysis module based on the analyzing of the implantable audio input relative to the external audio input. The new or updated weights/parameters can be used to update the training of the signal analysis module. The new or update weights/parameters can be transmitted to a signal analysis module of the internal component for use when invisible mode is triggered for the hearing device.

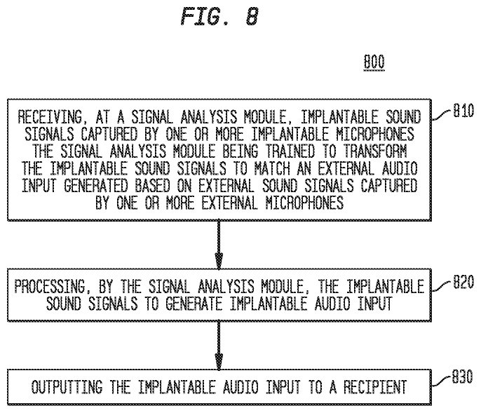

FIG. 8 is flowchart of an example method 800 of processing implantable sound signals to generate implantable audio input using a signal analysis module, in accordance with certain embodiments presented herein. At 810, a signal analysis module receives implantable sound signals captured by one or more implantable microphones (e.g., of a hearing device). The signal analysis module is trained to transform the implantable sound signals to substantially/approximately match an external audio input generated based on external sound signals captured by one or more external microphones (e.g., of the hearing device). For example, signal analysis module 362 can receive a sound signal captured by implantable sound sensors 160.

At 820, the signal analysis module processes the implantable sound signals to generate implantable audio input. At 830, the signal analysis module can output the implantable audio input to a recipient. For example, signal analysis module 362 can output the implantable audio input for processing by sound processing chain 440 and for delivery of the stimulation to the recipient to evoke perception of the sound signal.

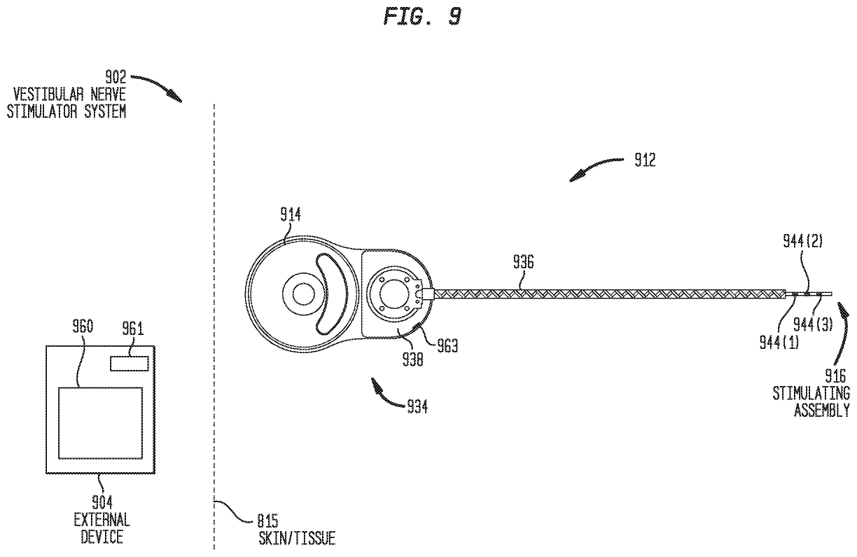

As previously described, the technology disclosed herein can be applied in any of a variety of circumstances and with a variety of different devices. Example devices that can benefit from technology disclosed herein are described in more detail in FIG. 9, below. As described below, the operating parameters for the devices described with reference to FIG. 9 can be configured using a fitting system. For example, the techniques described herein can be used to prioritize clinician tasks associated with configuring the operating parameters of wearable medical devices, such as a vestibular stimulator as described in FIG. 9. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 9 illustrates an example vestibular stimulator system 902, with which embodiments presented herein can be implemented. As shown, the vestibular stimulator system 902 comprises an implantable component (vestibular stimulator) 912 and an external device/component 904 (e.g., external processing device, battery charger, remote control, etc.). The external device 1004 comprises a transceiver unit 960 and external sensor 961. As such, the external device 904 is configured to transfer data (and potentially power) to the vestibular stimulator 912,

The vestibular stimulator 912 comprises an implant body (main module) 934, a lead region 936, and a stimulating assembly 916, all configured to be implanted under the skin/tissue (tissue) 915 of the recipient. The implant body 934 generally comprises a hermetically-sealed housing 938 in which RF interface circuitry, one or more rechargeable batteries, one or more processors, a stimulator unit, and an implantable sensor 963 are disposed. The implant body 934 also includes an internal/implantable coil 914 that is generally external to the housing 938, but which is connected to the transceiver via a hermetic feedthrough (not shown).

The stimulating assembly 916 comprises a plurality of electrodes 944(1)-(3) disposed in a carrier member (e.g., a flexible silicone body). In this specific example, the stimulating assembly 916 comprises three (3) stimulation electrodes, referred to as stimulation electrodes 944(1), 944(2), and 944(3). The stimulation electrodes 944(1), 944(2), and 944(3) function as an electrical interface for delivery of electrical stimulation signals to the recipient's vestibular system.

The stimulating assembly 916 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 912, the external device 904, and/or another external device, can be configured to implement the techniques presented herein. That is, the vestibular stimulator 912, possibly in combination with the external device 904 and/or another external device, can include a signal analysis module, as described elsewhere herein, that can be used to substantially match the signals output by implantable sensor to that of an external sensor (e.g., approximately match a response of the implantable sensor 963 within vestibular stimulator 912 to a response of the external sensor 961 of the external device 904)

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method comprising:
generating an external audio input based on sound signals captured by one or more external microphones;
generating, with a signal analysis module, an implantable audio input based on sound signals captured by one or more implantable microphones;
analyzing the implantable audio input relative to the external audio input; and
adjusting operation of the signal analysis module based on the analyzing of the implantable audio input relative to the external audio input.

2. The method of claim 1, wherein adjusting operation of the signal analysis module includes:
setting or updating weights associated with the signal analysis module.

3. The method of claim 1, wherein the signal analysis module is a deep neural network.

4. The method of claim 1, wherein the signal analysis module is trained to process the sound signals captured by the one or more implantable microphones to substantially match the external audio input.

5. The method of claim 1, wherein analyzing the implantable audio input relative to the external audio input includes computing an error function.

6. The method of claim 1, wherein the method is performed at an external device of a hearing device.

7. The method of claim 1, wherein the method is performed at an implantable device of a hearing device.

8. The method of claim 1, wherein the method is performed at a remote device in communication with a hearing device.

9. The method of claim 1, wherein generating the external audio input includes one or more of performing noise reduction, removing a noise floor, or adjusting a microphone directionality associated with the sound signals captured by one or more external microphones.

10. The method of claim 1, wherein generating the implantable audio input includes removing noises from the sound signals captured by one or more implantable microphones that are not present in the external audio input.

11. The method of claim 1, wherein generating the implantable audio input includes substantially matching a frequency response associated with the external audio input.

12. The method of claim 1, wherein generating the implantable audio input includes filling in missing frequency content in the sound signals captured by one or more implantable microphones.

13. The method of claim 1, further comprising determining that a hearing device is in a training mode.

14. A medical device, comprising:
one or more external sensors;
one or more implantable sensors;
a signal analysis module; and
one or more processors, wherein the one or more processors are configured to:
generate an external signal input based on signals captured by the one or more external sensors;
generate, by the signal analysis module, an implantable signal input based on signals captured by the one or more implantable sensors;
analyze the implantable signal input relative to the external signal input; and
adjust operation of the signal analysis module based on the analyzing of the implantable signal input relative to the external signal input.

15. The medical device of claim 14, wherein, when adjusting operation of the signal analysis module, the one or more processors are further configured to:
set or update weights associated with the signal analysis module.

16. The medical device of claim 14, wherein the signal analysis module is a deep neural network.

17. The medical device of claim 14, wherein the signal analysis module is trained to process the signals captured by the one or more implantable sensors to substantially match the external signal input.

18. The medical device of claim 14, wherein, when analyzing the implantable signal input relative to the external signal input, the one or more processors are further configured to compute an error function.

19. The medical device of claim 14, wherein, when generating the external signal input, the one or more processors are further configured to:
perform noise reduction, remove a noise floor, or adjust a microphone directionality associated with the signals captured by one or more external sensors.

20. The medical device of claim 14, wherein, when generating the implantable signal input, the one or more processors are further configured to remove noises from the signals captured by one or more implantable sensors that are not present in the external signal input.

21. The medical device of claim 14, wherein, when generating the implantable signal input, the one or more processors are further configured to substantially match a frequency response associated with the external signal input.

22. The medical device of claim 14, wherein, when generating the implantable signal input, the one or more processors are further configured to fill in missing frequency content in the signals captured by one or more implantable sensors.

23. The medical device of claim 14, wherein the one or more processors are further configured to determine that the medical device is in a training mode.

24. The medical device of claim 14, wherein the one or more implantable sensors and the one or more external sensors comprise sound sensors.

25. One or more non-transitory computer readable storage media comprising instructions that, when executed by a processor, cause the processor to:
receive, at a machine-learning device, implantable sound signals captured by one or more implantable microphones of a hearing device, the machine-learning device being trained to transform the implantable sound signals to substantially match an external audio input generated based on external sound signals captured by one or more external microphones;
process, by the machine-learning device, the implantable sound signals to generate implantable audio input; and
output the implantable audio input to a recipient of the hearing device.

26. The one or more non-transitory computer readable storage media of claim 25, wherein the machine-learning device is a deep neural network.

27. The one or more non-transitory computer readable storage media of claim 25, wherein, when processing the implantable sound signals, the processor is further configured to remove noises from the implantable sound signals that are not present in the external audio input.

28. The one or more non-transitory computer readable storage media of claim 25, wherein, when processing the implantable sound signals, the processor is further configured to substantially match a frequency response associated with the external audio input.

29. The one or more non-transitory computer readable storage media of claim 25, wherein, when processing the implantable sound signals, the processor is further configured to fill in missing frequency content in the implantable sound signals.
